(19) United States
(12) Patent Application Publication
(10) Pub. No.: US 2024/0033522 A1
(43) Pub. Date: Feb. 1, 2024

(54) Intra-Stimulus Recruitment Control

(71) Applicant: Saluda Medical Pty Ltd, Macquarie Park (AU)

(72) Inventors: Dean Michael Karantonis, Macquarie Park (AU); Peter Scott Vallack Single, Macquarie Park (AU); James Hamilton Wah, Macquarie Park (AU)

(73) Assignee: Saluda Medical Pty Ltd, Macquarie Park (AU)

(21) Appl. No.: 18/264,239

(22) PCT Filed: Feb. 9, 2022
(86) PCT No.: PCT/AU2022/050080

§ 371 Date: Aug. 3, 2023

(30) Foreign Application Priority Data
Feb. 9, 2021 (AU) 2021900311

(51) Int. Cl. A61N 1/36; A61N 1/02

(52) U.S. Cl. CPC A61N 1/36139; A61N 1/025; A61N 1/36153; A61N 1/36157; A61N 1/36062

(57) ABSTRACT

Recording evoked neural responses comprises delivering a stimulus from one or more stimulus electrodes to a neural pathway in order to give rise to an evoked compound action potential (ECAP) on the neural pathway. And, recording with measurement circuitry, during delivery of at least part of the stimulus, a neural compound action potential signal sensed at one or more sense electrodes. And, processing the recording of the neural compound action potential signal to determine at least one characteristic of the ECAP. At least one characteristic of the stimulus is then defined based on the determined at least one characteristic of the ECAP.

### TECHNICAL FIELD

The present invention relates to neurostimulation, and in particular the invention relates to the use of neural response measurements obtained during application of a stimulus in order to control ongoing application or cessation of that same stimulus.

### BACKGROUND OF THE INVENTION

There are a range of situations in which it is desirable to apply neural stimuli in order to give rise to an evoked compound action potential (ECAP). For example, neuromodulation is used to treat a variety of disorders including chronic pain, Parkinson's disease, and migraine. A neuromodulation system applies an electrical pulse to tissue in order to generate a therapeutic effect. When used to relieve chronic pain, the electrical pulse is applied to the dorsal column (DC) of the spinal cord, referred to as spinal cord stimulation (SCS). Neuromodulation systems typically comprise an implanted electrical pulse generator, and a power source such as a battery that may be rechargeable by transcutaneous inductive transfer. An electrode array is connected to the pulse generator, and is positioned in the dorsal epidural space above the dorsal column. An electrical pulse applied to the dorsal column by an electrode causes the depolarisation of neurons, and the generation of propagating action potentials. The fibres being stimulated in this way inhibit the transmission of pain from that segment in the spinal cord to the brain. To sustain the pain relief effects, stimuli are applied substantially continuously, for example at a frequency in the range of 10-100 Hz.

Neuromodulation may also be used to stimulate efferent fibres, for example to induce motor functions. In general, the electrical stimulus generated in a neuromodulation system triggers one or more neural action potentials, which then have either an inhibitory or excitatory effect, or otherwise electrically alters the neural conditions to achieve a desired effect. Inhibitory effects can be used to modulate an undesired process such as the transmission of pain, or excitatory effects may for example cause a desired effect such as the contraction of a muscle.

There are a range of circumstances in which it is desirable to obtain an electrical measurement of an ECAP evoked on a neural pathway by an electrical stimulus applied to the neural pathway. However, this can be a difficult task as an observed ECAP signal will typically have a maximum amplitude of a few tens of microvolts or less, whereas a stimulus applied to evoke the ECAP is typically several volts. Electrode artefact usually results from the stimulus, and manifests as a decaying output of several millivolts or hundreds of microvolts throughout the time that the ECAP occurs, presenting a significant obstacle to isolating the much smaller ECAP of interest. As the neural response can be contemporaneous with the stimulus and/or the stimulus artefact, ECAP measurements present a difficult challenge of implant design. In practice, many non-ideal aspects of a circuit lead to artefact, and as these mostly have a decaying exponential characteristic which can be of either positive or negative polarity, identification and elimination of sources of artefact can be laborious. A number of approaches have been proposed for recording an ECAP, including those of King (U.S. Pat. No. 5,913,882), Nygard (U.S. Pat. No. 5,758,651), Daly (US Patent Application No. 2007/0225767) and the present Applicant (U.S. Pat. No. 9,386,934).

Evoked responses are less difficult to detect when they appear later in time than the artefact, or when the signal-to-noise ratio is sufficiently high. The artefact is often restricted to a time of 1-2 ms after the stimulus and so, provided the neural response is detected after this time window, data can be obtained. This is the case in surgical monitoring where there are large distances between the stimulating and recording electrodes so that the neural response propagation time from the stimulus site to the recording electrodes exceeds 2 ms. However, neurostimulation implants are by necessity compact devices. To characterize responses evoked by a single implant such as responses from the dorsal columns to SCS, for example, high stimulation currents and close proximity between electrodes are required, and therefore the measurement process must overcome contemporaneous stimulus artefact directly, greatly exacerbating the difficulty of neural measurement.

Similar considerations can arise in deep brain stimulation where it can be desirable to stimulate a neural structure and immediately measure the evoked compound action potential produced in that structure before the neural response propagates elsewhere in the brain. Artefact remains a significant obstacle to measurement of neural responses proximal to the stimulus location, with the consequence that most neurostimulation implants do not take any measurements whatsoever of neural responses evoked by the implant's stimuli.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood that the element may be any one of the listed options, or may be any combination of two or more of the listed options.

### SUMMARY OF THE INVENTION

According to a first aspect the present invention provides a device for recording evoked neural responses, the device comprising:
a plurality of electrodes including one or more stimulus electrodes and one or more sense electrodes;
a stimulus source for providing a stimulus to be delivered from the one or more stimulus electrodes to a neural pathway in order to give rise to an evoked compound action potential (ECAP) on the neural pathway;
measurement circuitry for recording a neural compound action potential signal sensed at the one or more sense electrodes, the measurement circuitry being operable to record the neural compound action potential signal during delivery of at least part of the stimulus; and
a control unit configured to process the recording of the neural compound action potential signal to determine at least one characteristic of the ECAP, the control unit further configured to define at least one characteristic of the stimulus based on the determined at least one characteristic of the ECAP.

According to a second aspect the present invention provides a method for recording evoked neural responses, the method comprising:
delivering a stimulus from one or more stimulus electrodes to a neural pathway in order to give rise to an evoked compound action potential (ECAP) on the neural pathway;
recording with measurement circuitry, during delivery of at least part of the stimulus, a neural compound action potential signal sensed at one or more sense electrodes;
processing the recording of the neural compound action potential signal to determine at least one characteristic of the ECAP; and
defining at least one characteristic of the stimulus based on the determined at least one characteristic of the ECAP.

According to a further aspect the present invention provides a non-transitory computer readable medium for performing the method of the second aspect, comprising instructions which, when executed by one or more processors, causes performance of the steps of the second aspect.

The at least one characteristic of the ECAP is preferably selected or predetermined as being a characteristic which reflects an efficacy of the stimulus. For example, the least one characteristic may reflect a therapeutic efficacy of neural recruitment achieved by the stimulus. The at least one characteristic of the ECAP may comprise a binary characteristic, such as a presence or absence of an ECAP or a comparison of the ECAP to a threshold. Additionally or alternatively, the at least one characteristic may comprise a gradated or scalar indication of an observed feature of the recording. The at least one characteristic of the ECAP may comprise one or more of: an indication as to whether or not the stimulus has recruited an ECAP; a measure of ECAP onset delay time; a measure of ECAP slope; a measure of ECAP amplitude such as an instantaneous amplitude of the recording, an averaged amplitude of the recording over 2 or more digital samples, and/or an ECAP peak amplitude; a measure of ECAP duration such as ECAP peak width, ECAP zero crossing spacing, or ECAP half height width; a measure of ECAP spectral components such as may be obtained by fast Fourier transform (FFT); or the like. The at least one characteristic of the action potential may comprise any such characteristic of a late response arising contemporaneously with the stimulus.

In some embodiments, recording of the neural compound action potential signal may cease upon detection of a threshold, or may cease when the stimulus ceases, or may cease at a time defined relative to such occasions. Alternatively, recording of the neural compound action potential signal may continue after the step of defining at least one characteristic of the stimulus is completed, for example in order to retrieve a lengthier recording of improved quality, for use by secondary processes such as a supervisory process providing feedback improvements of the process for determining the at least one characteristic of the evoked action potential.

The present invention in some embodiments may thus provide for the at least one characteristic of the ECAP to be determined prior to cessation of the stimulus, and may further provide for the determined the at least one characteristic of the ECAP to be used to control the manner in which a remainder of the stimulus is applied. That is, in some embodiments the present invention provides for characteristic(s)) of the stimulus to be controlled by observing the neural response which the stimulus itself has generated.

In some embodiments of the invention, the at least one characteristic may comprise an observation that the ECAP recording has reached a threshold amplitude. In such embodiments, defining the at least one characteristic of the stimulus may comprise immediately ceasing the stimulus upon observing that the ECAP recording has reached the threshold amplitude. Alternatively, defining the at least one characteristic of the stimulus may comprise ceasing the stimulus a predetermined time after observing that the ECAP recording has reached the threshold amplitude.

In some embodiments of the invention, defining at least one characteristic of the stimulus based on the determined efficacy of the stimulus may comprise altering or defining at least one stimulus parameter in order to control the stimulus to deliver a desired dose of neural recruitment. For example the at least one parameter may be adjusted in a manner so as to control the amount of charge delivered to the tissue by the stimulus. For example a duration of the stimulus may be adjusted on the basis of the determined efficacy of the stimulus. Additionally or alternatively an amplitude, intensity, voltage, current and/or morphology of the stimulus may be adjusted on the basis of the determined efficacy of the stimulus.

In some embodiments of the invention, defining at least one characteristic of the stimulus based on the determined efficacy of the stimulus may comprise altering or defining a number of pulses of the stimulus. For example, based on the first controlled pulse, a series of subsequent pulses may be generated, wherein a relationship between the first and subsequent pulses may be that a pulse shape is identical, or that subsequent pulses decay in either pulse width or amplitude at some rate for a number N of pulses, or that subsequent pulses are different to the first pulse but all the same as each other.

In some embodiments, defining at least one characteristic of the stimulus based on the determined at least one characteristic of the evoked action potential may result in the delivered stimulus not being charge balanced. Accordingly, in such embodiments charge balancing may be effected by thereafter delivering charge balancing in a sub-threshold manner, or by active charge recovery using a charge recovery pulse of the same shape as the stimulus, or by using a charge recovery pulse of reduced amplitude and longer duration than the stimulus. Alternatively, charge balancing may in some embodiments be effected via passive charge recovery. Additionally or alternatively, charge balancing may in some embodiments be effected by delivering a cathodic phase prior to an anodic phase so that characteristics of both phases can be adjusted in order to optimise neural recruitment dose as well as maintain charge balancing.

Some embodiments of the invention may further provide for jointly considering both (a) a during-stimulation ECAP measurement and (b) at least one previous ECAP measurement. Such embodiments may for example provide for improved signal-to-noise-ratio (SNR) assessment of slowly changing stimulus transfer function characteristics, while also providing for rapid assessment of rapidly changing stimulus transfer function characteristics at a lower SNR. For example, when a patient coughs such embodiments may provide for a stimulus to be rapidly cut short upon detection of an unexpected ECAP, even if the detection has a poor SNR. Such embodiments may thus serve as a supplemental function to conventional closed loop control.

In some embodiments of the invention, measuring the neural response may be done on the same electrode as is delivering the stimulation. That is to say that in such embodiments, the one or more stimulus electrodes also serve as the one or more recording electrodes. Such embodiments are advantageous in allowing for most rapid detection of any recruited neural response by eliminating any neural propagation delay, noting that the finite conduction velocity of neural responses necessarily results in the neural response arising on more distant electrodes at a later time.

In alternative embodiments, measuring the neural response may be effected by way of a sense electrode which is a non-stimulating electrode near the stimulus electrode. To permit observation of a neural response to commence prior to cessation of the stimulus, the sense electrode(s) may be positioned at a distance from the stimulus electrodes which is less than 120 mm, preferably less than 100 mm, more preferably less than 80 mm, and most preferably less than 60 mm. The sense electrodes may be mounted on an electrode lead upon which the stimulus electrodes are mounted.

In some embodiments of the invention, measuring the neural response and defining at least one characteristic of the stimulus may be programmed to occur only at a certain interval, amongst periods of open loop operation or non-adaptive operation. For example, measuring the neural response and defining at least one characteristic of the stimulus may be triggered to occur based on one or more factors, such as a physiological trigger, activity of the patient or inputs from other sensors.

Some embodiments of the invention may apply multi-phase stimulus control, so as configure a later phase of the stimulus based upon measurements of neural activation obtained in response to a previous phase of the stimulus.

Additionally or alternatively, some embodiments of the invention may also apply multi-stimulus feedback control, so as also configure the stimulus based upon measurements of neural activation obtained in response to a previous stimulus.

In some embodiments of the invention, the measurement circuitry may be blanked for some portion or portions of a period in which the stimulus crosstalk voltage arises, whereby during blanking some or all of the measurement circuitry is disconnected from the sense electrodes, whereby during blanking an output of the measurement circuitry does not carry useful measurement information but also does not suffer from stimulus crosstalk. For example, the measurement circuitry may be blanked during one or more stimulus transients, referred to herein as transient blanking. Transient blanking may be imposed during one or more of an onset of a stimulus phase and cessation of a stimulus phase, for one or more anodic stimulus phase(s) and/or for one or more cathodic stimulus phase(s). Transient blanking may be imposed for example for a period in the range of 10-50 μs either side of a stimulus transient. Noting that a stimulus phase width may be around 0.1-1 ms, such embodiments may thus provide for the measurement circuitry to be unblanked for 80-95% of the duration of each stimulus phase, while being blanked to avoid exposure to stimulus transients, allowing for evoked neural responses to be observed for a significant portion of the stimulus period while avoiding non-linearity, clipping or saturation of the measurement circuitry.

To permit observation of a neural response to commence prior to cessation of the stimulus, the measurement circuitry is preferably unblanked or activated immediately following a stimulus feature which is expected to cause neural activation, such as the leading edge of a cathodic portion of the stimulus. For example, the measurement circuitry may be unblanked or activated within 50 μs, more preferably 20 μs, more preferably 10 μs, after such a stimulus feature.

Some embodiments of the invention may provide for a stimulus protocol to be applied whereby stimuli are delivered at high frequency and low current, in which a single stimulation is not expected to elicit a neural response but the temporal summation of several stimulations is intended to recruit an ECAP. Such embodiments of the invention may provide for the stimulus protocol to be halted once an ECAP is observed or once the ECAP amplitude, peak width or the like reaches a threshold.

In some embodiments of the invention, the detection/measurement of the ECAP may be carried out in parallel on more than one recording electrode, to improve signal detection due to the spatial and temporal variations which will occur.

Some embodiments of the invention may provide for a stimulus intensity, such as stimulus current and/or stimulus voltage, to be progressively raised from a sub-threshold level so as to search for an ECAP recruitment threshold.

Some embodiments of the invention may compare the ECAP intensity to an overstimulation threshold and, upon the ECAP being observed to exceed the overstimulation threshold, may immediately trigger cessation of the stimulus.

The neuromodulation may comprise spinal cord stimulation, sacral nerve stimulation, deep brain stimulation (DBS), vagus nerve stimulation or other form of neuromodulation.

The method may be applied in respect of a single stimulus applied in isolation, or in respect of multiple stimuli applied repeatedly, sporadically or continuously, such as at less than 10 Hz, at tens of Hz or at hundreds of Hz.

Some embodiments may comprise DBS monitoring of beta band oscillations, whereby the intra-stimulus response is measured continuously. For example DBS may be applied at tens or hundreds of Hz, and beta band oscillations variations may be computed and compared to changes of stimulus intensity or frequency or the like.

The stimulus may comprise a continuous or piecewise continuous waveform, wherein responses evoked by the continuous waveform can be used to adjust ongoing application of the waveform.

### BRIEF DESCRIPTION OF THE DRAWINGS

An example of the invention will now be described with reference to the accompanying drawings, in which:

FIG. 1 schematically illustrates an implanted spinal cord stimulator in accordance with one embodiment of the invention;

FIG. 2 is a block diagram of the implanted neurostimulator of FIG. 1;

FIG. 3 is a schematic illustrating interaction of the implanted stimulator with a nerve;

FIG. 4 depicts simplified waveforms of a prior art blanked ECAP recording system;

FIG. 5 depicts the sequential nature of prior art closed loop neuromodulation;

FIG. 6 depicts a method for single stimulus closed loop neuromodulation in accordance with an embodiment of the invention;

FIG. 7 is a flowchart depicting the operation of intra-stimulus ECAP detection and feedback control in accordance with one embodiment of the invention;

FIG. 8 depicts component waveforms as may arise in the embodiment of FIG. 7; and

FIG. 9 is a plot of results from experimental implementation of a system for recording during a stimulus.

### DESCRIPTION OF THE PREFERRED EMBODIMENTS

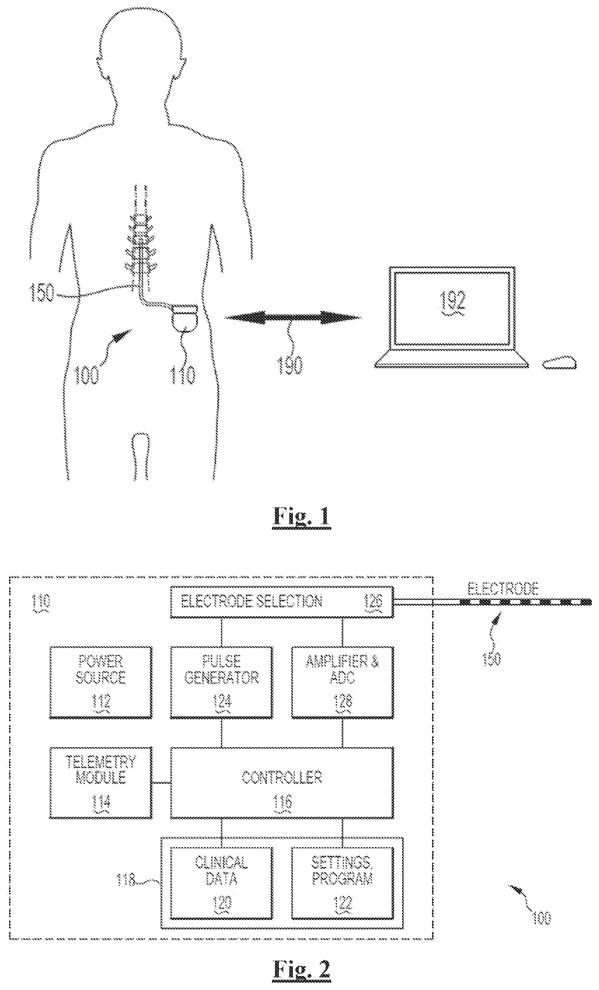

FIG. 1 schematically illustrates an implanted spinal cord stimulator 100. Stimulator 100 comprises an electronics module 110 implanted at a suitable location in the patient's lower abdominal area or posterior superior gluteal region, and an electrode assembly 150 implanted within the epidural space and connected to the module 110 by a suitable lead. Numerous aspects of operation of implanted neural device 100 are reconfigurable by an external control device 192. Moreover, implanted neural device 100 serves a data gathering role, with gathered data being communicated to external device 192 via any suitable transcutaneous communications channel 190.

FIG. 2 is a block diagram of the implanted neurostimulator 100. Module 110 contains a battery 112 and a telemetry module 114. In embodiments of the present invention, any suitable type of transcutaneous communication 190, such as infrared (IR), electromagnetic, capacitive and inductive transfer, may be used by telemetry module 114 to transfer power and/or data between an external device 192 and the electronics module 110. Module controller 116 has an associated memory 118 storing patient settings 120, control programs 122 and the like. Controller 116 controls a pulse generator 124 to generate stimuli in the form of current pulses in accordance with the patient settings 120 and control programs 122. Electrode selection module 126 switches the generated pulses to the appropriate electrode(s) of electrode array 150, for delivery of the current pulse to the tissue surrounding the selected electrode(s). Measurement circuitry 128 is configured to capture measurements of neural responses sensed at sense electrode(s) (also referred to as measurement electrodes or recording electrodes) of the electrode array as selected by electrode selection module 126.

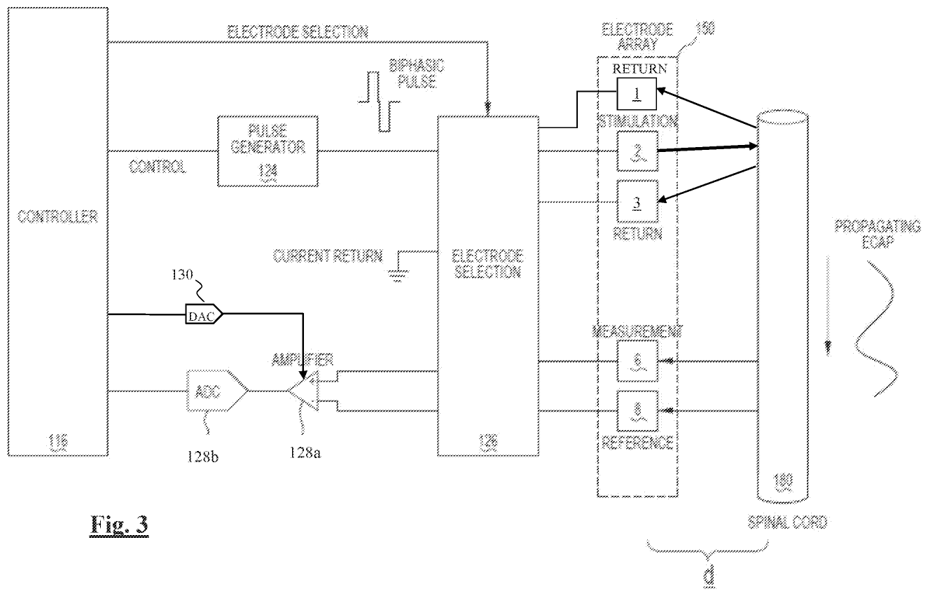

FIG. 3 is a schematic illustrating interaction of the implanted stimulator 100 with a nerve 180, in this case the spinal cord however alternative embodiments may be positioned adjacent any desired neural tissue including a peripheral nerve, visceral nerve, parasympathetic nerve or a brain structure. The pulse generator 124 produces a suitable stimulus pulse, which in FIG. 3 is shown as a biphasic pulse, although alternative embodiments of the invention may utilise a triphasic pulse or other multiphasic pulse for example in accordance with the teachings of in International Patent Publication No. WO 2017/219096 by the present applicant, the content of which is incorporated herein by reference. Electrode selection module 126 selects a stimulation electrode 2 of electrode array 150 to deliver the electrical current pulse to surrounding tissue including nerve 180, and selects return electrodes 1 and 3 for stimulus current recovery to maintain a zero net charge transfer. In this manner electrode selection module 126 effects tripolar stimulation via electrodes 1, 2, 3, for example in accordance with the teachings of the above-noted WO 2017/219096, and/or in accordance with the teachings of International Patent Application Publication No. WO 2020/082118 by the present applicant, the content of which is incorporated herein by reference. Alternative embodiments may utilise bipolar stimulation by use of two electrodes.

Delivery of an appropriate stimulus to the nerve 180 evokes a neural response comprising a compound action potential which will propagate along the nerve 180 as illustrated, for therapeutic purposes which in the case of a spinal cord stimulator for chronic pain might be to create paraesthesia at a desired location. To this end the stimulus electrodes are used to deliver stimuli at any therapeutically suitable time(s) or frequency(ies). To fit the device, a clinician typically applies stimuli of various configurations which seek to produce a sensation that is experienced by the user as a paraesthesia, or generally to provide a desirable therapy. When a stimulus configuration is found which evokes paraesthesia, which is in a location and of a size which is congruent with the area of the user's body affected by pain, the clinician nominates that configuration for ongoing use.

The device 100 is further configured to sense the existence and intensity of compound action potentials (CAPs) propagating along nerve 180, whether such CAPs are evoked by the stimulus from electrodes 2 and 4, or otherwise evoked. To this end, any electrodes of the array 150 may be selected by the electrode selection module 126 to serve as measurement electrode 6 and measurement reference electrode 8. Signals sensed by the measurement electrodes 6 and 8 are passed to measurement circuitry comprising one or more amplifiers 128a, which for example may operate in accordance with the teachings of International Patent Application Publication No. WO 2012/155183 by the present applicant, the content of which is incorporated herein by reference, and/or the measurement circuitry may operate in accordance with the teachings of International Patent Application Publication No. WO 2021/232091, the content of which is incorporated herein by reference. The output of the amplifier(s) 128a is then digitised by analog to digital converter 128b and passed to the controller 116. Digital-to-analog converter 130, which receives digital input from controller 116 and converts the received digital input into an analog output, modifies the operation of amplifier 128a as described in International Patent Application Publication No. WO 2021/232091. Nevertheless, artefact remains a significant obstacle to measurement of neural responses proximal to the stimulus location. The present Applicant has previously presented a model of the neurostimulation environment, in International Patent Application Publication No. WO 2020/082126, the contents of which are incorporated herein by reference.

Recording evoked compound action potentials thus requires the delivery of an electrical stimulus, and the recording of electrical potentials produced by the stimulated nerves. This is challenging because the evoked potentials can be much smaller than the stimuli, for example around six orders of magnitude smaller. Unless special measures are taken the stimulus, and its after-effects such as stimulus artefact, obscures the response. For example, in spinal cord stimulation, where a distance d between the electrode array 150 and the nerve 180 can be several millimetres, a therapeutically optimal stimulus applied by electrodes 1, 2, 3 can be on the order of 10 volts, while the evoked potential observed on the measurement electrodes 6, 8 can be on the order of 10 microvolts. The evoked responses generally must be recorded very quickly after the stimulus, as the duration of the evoked responses is typically quite short, the recording electrodes 6,8 are close to the stimulus electrodes 1, 2, 3 due to the limited size of the implanted device, and the conduction velocity of the nerve 180 is quite high (e.g. in the range 15-70 m·s⁻¹). As a result, depending on the electrode configuration and the conduction velocity of the nerves stimulated, a 1.5 millisecond duration of evoked responses is typical. Building a system to directly digitise a waveform with this dynamic range is impractical; in this example, resolving the ECAP to just 4 bits of resolution would require a signal chain and ADC with no less than 24 bits of effective resolution, sampling on the order of 1 kHz. This is not practical with present technology, particularly for a compact implantable device with limited power budget.

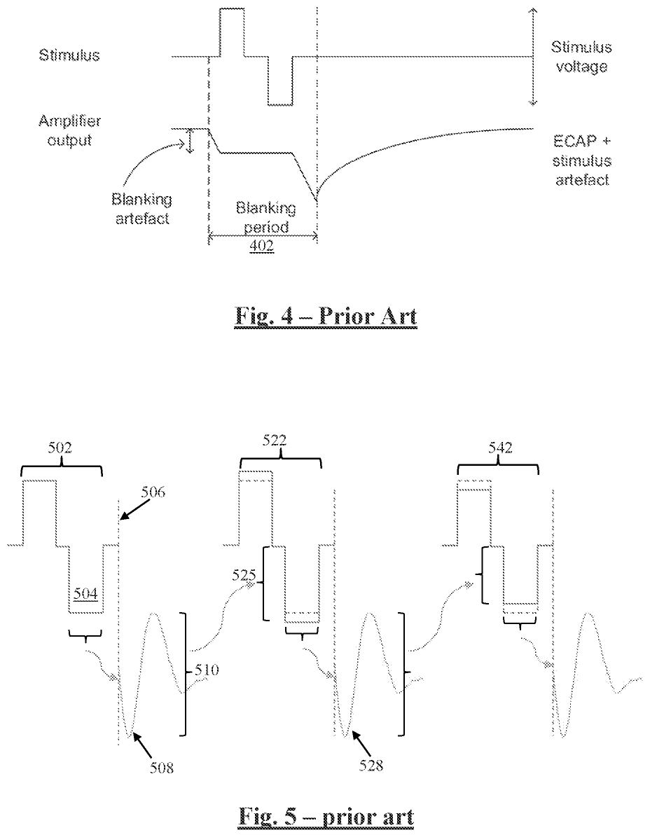

Existing ECAP amplifiers avoid this problem using blanking. Blanking involves disconnecting the recording amplifier(s) 128a, which have high gain, from the recording electrodes 6, 8 during the stimulus and for a short period immediately thereafter. Shortly after the stimulus is completed, the amplifiers 128a are reconnected, and thereafter the signal from the recording electrodes 6, 8 is recorded, including the ECAP and any extant artefact. The blanking period must be sufficiently long that the extant artefact has reduced sufficiently after cessation of the stimulus that the amplifiers 128a are not saturated. However, a consequence of blanking is that any component of neural response which occurs during the blanking period is not recorded. Depending on the length of the blanking period, the conduction velocity of the nerve fibres recruited by the stimulus, and the physical extent (e.g. length) of the recording electrode array 150, the imposition of such a blanking period can result in a significant loss of information. FIG. 4 depicts simplified waveforms of such a blanked ECAP recording system. For the blanking period 402 surrounding the stimulus, the amplifier input is disconnected from the recording electrodes, so the amplifier output carries no useful signal. After reconnection, the amplifier output takes some time to come out of blanking. Only after this time does the amplifier output actually represent the ECAP (if any) and the stimulus artefact present at the recording electrodes 6,8.

As a consequence of the blanking approach to ECAP measurement, existing approaches to closed-loop (CL) neurostimulation require that a stimulus pulse is delivered to tissue and then, after completion of the stimulus, a neural response to the delivered pulse is measured. A controller then adjusts a subsequent stimulus pulse's intensity (current, charge, etc.), based on this measurement obtained from the preceding stimulus. This process is illustrated in FIG. 5, wherein the parameters of each stimulus are defined on the basis of the neural response evoked by the preceding stimulus. In more detail, a first stimulus 502 is applied, the stimulus 502 comprising a cathodic portion 504 which evokes a neural response. The stimulus 502 is necessarily configured so that the stimulus 502 concludes quickly, prior to a time 506, so that the evoked neural response can be recorded. Thus, a first recording 508 of a first ECAP is obtained after the stimulus 502 has concluded. Characteristics of the first ECAP recording 508, such as the ECAP amplitude 510, are then used by the closed loop controller to define the parameters of a subsequent second stimulus 522, such as the amplitude 525 of the second stimulus 522. The second stimulus 522 is then applied, after which a second recording 528 of a second ECAP can be obtained, and the process can be repeated to define a third stimulus 542, and so on, so as to effect closed loop control of neuromodulation.

However, a neural response evoked by a first stimulus, such as stimulus 502, can swiftly become irrelevant to understanding or predicting the neural response which will be evoked by a subsequent stimulus, such as stimulus 522. There are a range of situations where rapid changes can occur in the stimulus transfer function, the stimulus transfer function being the relationship of an applied stimulus intensity to the resulting evoked neural response. The stimulus transfer function can rapidly change, for example when the patient coughs or sneezes. This imposes practical limits on how swiftly the next adjusted stimulus pulse is required to be delivered, because ideally the stimulus frequency is sufficiently high to allow the device to possess a suitably fast response time to cater for the fastest expected changes in electrode-cord distance, and to cater for the concomitant rapid changes in the stimulus transfer function. Such conventional closed loop neuromodulation approaches are thus confined to operation at a stimulus frequency greater than 10-20 Hz, referred to hereinafter as the conventional closed loop minimum frequency. The system will suffer a reduced ability to adapt to a changing stimulus transfer function, and will thus deliver suboptimal performance if the stimulation frequency is less than the conventional closed loop minimum frequency.

Regarding spinal cord stimulation (SCS), it is unknown what the lowest rate (periodic or otherwise) of stimulus pulses is, required to deliver an efficacious therapy. Such a rate is likely to differ between patients. Since SCS generally elicits a percept, there is a necessity to deliver a regular pulse train in order for the therapy to be tolerated by the patient. For neural targets other than the spinal cord, there is also generally a lack of fundamental knowledge around the lowest efficacious stimulation rate. For those therapies that do not elicit a percept, there may be more scope, depending on that therapy's mechanism of action, for a lower stimulation rate than is currently offered as a treatment.

In general, it can be observed that if, for a given patient, the lowest efficacious stimulation rate is less than the conventional closed loop minimum frequency, then during closed loop operation a conventional closed loop device must operate at a higher frequency than is therapeutically necessary, merely in order to sustain the stimulus rate above the conventional closed loop minimum frequency. Thus, in such cases excess power must be consumed to deliver this higher stimulus rate, for no therapeutic benefit. And, power consumption is a critical factor in battery-powered implanted devices. In the particular case where a neuromodulation therapy does not evoke a side effect related to stimulus rate, such as a percept such as paraesthesia, then reducing the stimulus rate below the conventional closed loop minimum frequency towards the lowest efficacious stimulation rate would bring the benefit of reduced power consumption, with no therapeutic disadvantage. Even in cases where the therapy does elicit a side effect related to stimulus rate, such as a percept such as paraesthesia, the therapeutic disadvantage of reducing the stimulus rate can be clinically balanced against the power consumption savings.

Accordingly, the present disclosure recognises that in therapies where the lowest stimulation rate which is required in order to be suitably efficacious (whilst avoiding side-effects or otherwise maintaining patient acceptance) is lower than the rate required by existing closed-loop algorithms, there is an opportunity to save power by delivering stimulation at that lower rate. However, there is still a need to control the dose of therapy delivered, in order to avoid dose-related side-effects. So simply lowering the stimulation rate in an open loop stimulation mode is not necessarily an option for optimising the therapeutic outcome.

The following embodiments recognise that an approach to solving this problem is by measuring the neural response whilst a first stimulus is delivered, ascertaining what neural response the first stimulus itself is generating or has generated, and controlling some aspect of the first stimulus based on that neural response. For example, the amount of charge delivered to the tissue by the first stimulus may be controlled in this way.

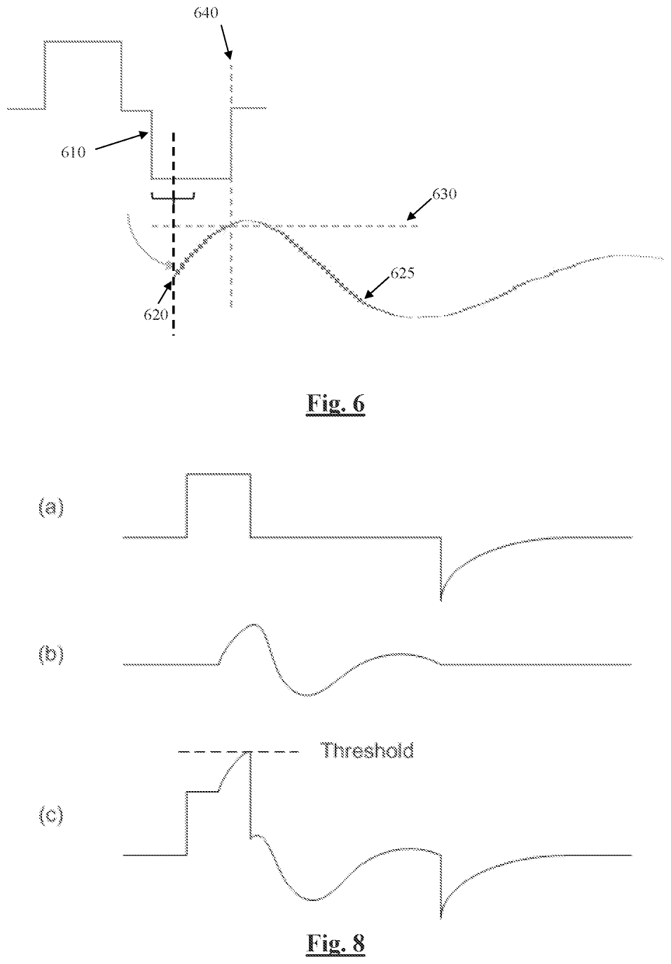

In this manner, these embodiments of the invention decouple (a) the necessity of having a train of pulses from (b) controlling the dose based on the neural response (of the previous pulse). Instead, it becomes possible to have a temporally-independent dose-controlled stimulation pulse, or in other words, a single-stimulus ECAP-controlled therapy. One embodiment of this is shown in FIG. 6. This depicts single-stimulus ECAP-controlled therapy, in which a cathodic pulse is commenced at time 610. This causes the neural tissue to start to depolarise, and after a short time an ECAP 625 will become measurable at time 620. This is observed by a controller in real time and, once the controller detects that the amplitude of the ECAP 625 reaches a defined amplitude 630, the controller uses this as a trigger to cease the input stimulus pulse at 640. Any unbalanced charge can be recovered after the measurement is complete. In other embodiments, an alternative or additional control feature other than threshold 630 may be implemented, such as comparing a rate of rise of an initial portion of the ECAP 625 to a threshold, and reducing or ceasing the stimulus if the rate of rise exceeds the threshold.

In particular, the stimulus cessation time 640 is not known when application of the stimulus commences. Instead the stimulus cessation time is determined on the fly in response to the initial portion of the observed neural response 625, after time 620 and before time 640. This approach thus allows for the stimulus to be abbreviated if the neural recruitment is larger than desired, or for the stimulus to be extended and/or altered to thereafter be of greater amplitude/intensity if the neural recruitment is less than desired. More than one such alteration to the stimulus may occur prior to cessation of the stimulus.

In the embodiment of FIG. 6, removing the need for stimulus control to be predicated on a previous stimulus removes the requirement to have a pulse train of frequency greater than or equal to the conventional closed loop minimum frequency in order to effect closed loop operation. Thus closed loop operation, involving stimulus control in response to observed neural recruitment, can still be provided at low frequencies below the conventional closed loop minimum frequency by way of the embodiment of FIG. 6 or other similar embodiments. The freedom thus achieved may for example be exploited in order to reduce power consumption.

Further embodiments similar to that of FIG. 6 may distinguish the electrical pulses generated by the system as being either a therapy pulse or an ECAP detection pulse. The therapy pulse may be delivered to the anatomy of the patient based on the dose requirements of the patient. The ECAP detection pulse may be delivered to measure an ECAP and take remedial actions if necessary. In some cases, the therapy pulse may act as an ECAP detection pulse.

An ECAP detection pulse of the type shown in FIG. 6 may be programmed to occur at a certain interval or may be triggered based on one or more factors. The factors triggering ECAP recording may include, for example: a physiological trigger such as heart rate, blood pressure; neural activity such as slow responses or doublets; time of day; activity of the patient; location of the patient such as proximity to a home logging device; posture of the patient; and inputs from sensors such as accelerometers, heart rate monitors, sleep monitors, ECG monitors and EEG monitors.

This technique is thus particularly useful in applications where a therapeutic requirement for the stimulus frequency is low. Nevertheless, the system may be configured to work for virtually any practical stimulus rate including rates higher than the conventional closed loop minimum frequency. This method of stimulation may result in increased battery life due to low battery consumption.

The ECAP recordings used for the present invention may be obtained by any suitable technique for recording neural response data during some or all of the blanking period 402. For example, the measurement circuitry may operate in accordance with the teachings of the aforementioned International Patent Application Publication No. WO 2021/232091, or any other suitable techniques. Embodiments of the present invention thus provide for using neural response recordings obtained during some or all of the stimulus itself for the purpose of improved neuromodulation control.

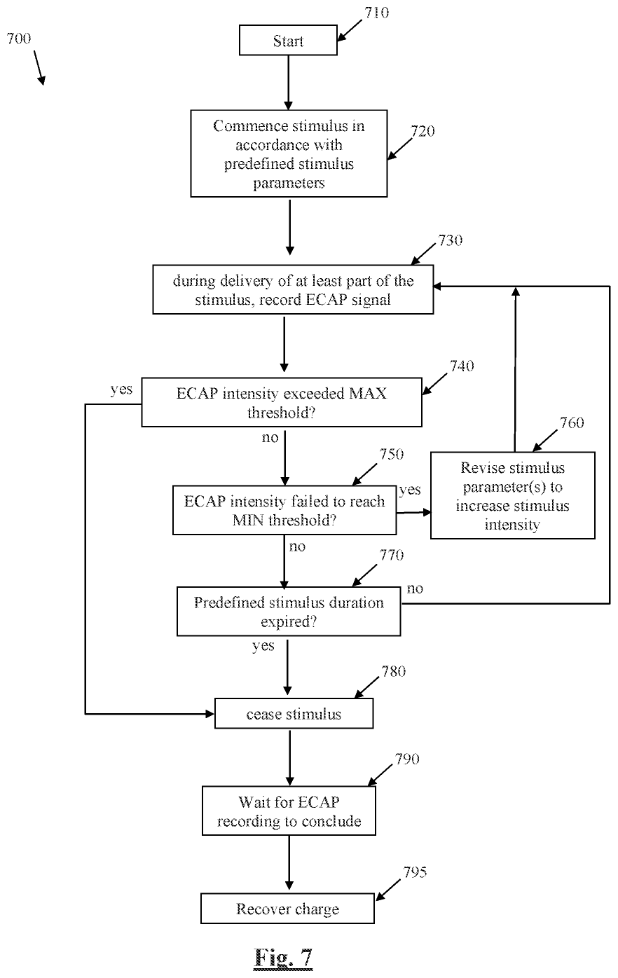

FIG. 7 is a flowchart illustrating a method 700 of intra-stimulus ECAP detection and feedback control in accordance with one embodiment of the present invention. The method 700 may be carried out by the controller 116 as configured by the control programs 122. The method 700 starts at step 710, then proceeds to step 720, which commences the delivery of stimulus according to predefined stimulus parameters. Step 730 then records an ECAP signal during at least part of the stimulus delivery of step 720. Step 740 then checks whether the ECAP intensity (e.g. amplitude) in the recorded signal exceeds a threshold MAX which may correspond to an overstimulation threshold. If so, the method 700 proceeds to step 780 which ceases the stimulus commenced at step 720. In some embodiments, step 780 ceases the stimulus a predetermined time after observing that the ECAP recording has reached the threshold amplitude at step 740.

If not, step 750 checks whether the ECAP intensity (e.g. the instantaneous amplitude) fails to reach a minimum threshold MIN. If so, step 760 revises one or more of the stimulus parameters, such as stimulus pulse current or voltage, to increase the intensity of the stimulus commenced at step 720. If not, the method 700 proceeds directly to step 770 which awaits the expiry of a predefined stimulus duration. If the duration has not yet expired, the method returns to step 730 to continue recording the ECAP signal. Once the duration has expired, step 780 ceases the stimulus commenced at step 720. Step 790 then waits for the ECAP recording commenced at step 730 to conclude, which may occur after a predefined delay after ceasing the stimulus. Step 795 then recovers any imbalance of charge that occurred as a result of ceasing the stimulus before the predefined duration had expired. Charge balancing may be delivered by delivering a stimulus pulse in a sub-threshold manner, or by active charge recovery using a charge recovery pulse of the same shape as the stimulus, or by using a charge recovery pulse of reduced amplitude and longer duration than the stimulus. Alternatively, charge balancing may in some embodiments be effected via passive charge recovery. Additionally or alternatively, charge balancing may in some embodiments be effected by delivering a cathodic phase prior to an anodic phase so that characteristics of both phases can be adjusted in order to optimise neural recruitment dose as well as maintain charge balancing.

In alternative embodiments, characteristics of the ECAP other than instantaneous amplitude may be determined and compared with one or more thresholds to determine whether to cease stimulus. In some embodiments, the characteristic is a binary characteristic, such as a presence or absence of an ECAP in the recording, that is, an indication as to whether or not the stimulus has recruited an ECAP. In another such embodiment, the at least one characteristic may comprise an indication that the ECAP in the recording has reached a threshold amplitude. In other embodiments, the at least one characteristic may comprise a gradated or scalar indication of an observed feature of the recording. The at least one characteristic of the action potential may comprise one or more of: a measure of ECAP onset delay time; a measure of ECAP slope; an averaged amplitude or trend line of the recording over two or more digital samples; an ECAP peak amplitude; a measure of ECAP duration such as ECAP peak width, ECAP zero crossing spacing, or ECAP half height width; a measure of ECAP spectral components such as may be obtained by fast Fourier transform (FFT) or the like. The at least one characteristic of the ECAP may comprise any such characteristic of a late response arising contemporaneously with the stimulus.

In some embodiments, recording of the neural compound action potential signal may cease upon detection that the ECAP intensity exceeds a threshold at step 740, or when the stimulus ceases at step 780, or at a time defined relative to such occasions. Alternatively, recording of the neural compound action potential signal may continue after the step of defining at least one characteristic of the stimulus (such as by ceasing the stimulus) is completed, for example in order to retrieve a lengthier recording of improved quality, for use by secondary processes such as a supervisory process providing feedback improvements of the process for determining the at least one characteristic of the evoked compound action potential.

In some embodiments, defining or revising at least one characteristic of the stimulus based on the determined efficacy of the stimulus may comprise defining or revising at least one stimulus parameter in order to control the stimulus to deliver a desired dose of neural recruitment. For example, the at least one parameter may be adjusted in a manner so as to control the amount of charge delivered to the tissue by the stimulus. For example, a duration of the stimulus may be adjusted on the basis of the determined efficacy of the stimulus. Additionally, or alternatively, an amplitude, intensity, voltage, current and/or morphology of the stimulus may be adjusted on the basis of the determined efficacy of the stimulus.

In some embodiments, defining at least one characteristic of the stimulus based on the determined efficacy of the stimulus may comprise defining or revising a number of pulses of the stimulus. For example, based on the first controlled pulse, a series of subsequent pulses may be generated, wherein a relationship between the first and subsequent pulses may be that a pulse shape is identical, or that subsequent pulses decay in either pulse width or amplitude at some rate for a number N of pulses, or that subsequent pulses are different to the first pulse but all the same as each other.

Some embodiments may further provide for jointly considering both (a) a during-stimulation ECAP measurement, as in FIG. 7, and (b) at least one previous ECAP measurement. Such embodiments configure the stimulus based upon measurements of neural activation obtained in response to a previous stimulus. Such embodiments may for example provide for improved signal-to-noise-ratio (SNR) assessment of slowly changing stimulus transfer function characteristics, while also providing for rapid assessment of rapidly changing stimulus transfer function characteristics at a lower SNR. For example, when a patient coughs such embodiments may provide for a stimulus to be rapidly cut short upon detection of an unexpected ECAP, even if the detection has a poor SNR. Such embodiments may thus serve as a supplemental function to conventional closed loop control.

In some embodiments, measuring the neural response may be done on the same electrode on which the stimulation is delivered. That is to say that in such embodiments, one or more of the one or more stimulus electrodes also serve as one or more of the one or more recording electrodes. Such embodiments are advantageous in allowing for most rapid detection of any recruited neural response by eliminating any neural propagation delay, noting that the finite conduction velocity of neural responses necessarily results in the neural response arising on more distant electrodes at a later time.

In alternative embodiments, measuring the neural response may be effected by way of a sense electrode which is a non-stimulating electrode near the stimulus electrode, as illustrated in FIG. 3. To permit observation of a neural response to commence prior to cessation of the stimulus, the sense electrode(s) may be positioned at a distance from the stimulus electrodes which is less than 120 mm, preferably less than 100 mm, more preferably less than 80 mm, and most preferably less than 60 mm. The sense electrodes may be mounted on an electrode lead upon which the stimulus electrodes are mounted.

In some embodiments, measuring the neural response and defining at least one characteristic of the stimulus may be programmed to occur only at a certain interval, amongst periods of open loop operation or non-adaptive operation. For example, measuring the neural response and defining at least one characteristic of the stimulus may be triggered to occur based on one or more factors, such as a physiological trigger, activity of the patient or inputs from other sensors such as an accelerometer.

Some embodiments invention may apply multi-phase stimulus control, so as configure a later phase of the stimulus based upon measurements of neural activation obtained in response to a previous phase of the stimulus.

In some embodiments, the measurement circuitry 128 may be blanked for some portion or portions of a period in which the stimulus crosstalk voltage arises, whereby during blanking some or all of the measurement circuitry 128 is disconnected from the sense electrodes, whereby during blanking an output of the measurement circuitry 128 does not carry useful measurement information but also does not suffer from stimulus crosstalk. For example, the measurement circuitry may be blanked during one or more stimulus transients, referred to herein as transient blanking. Transient blanking may be imposed during one or more of an onset of a stimulus phase and cessation of a stimulus phase, for one or more anodic stimulus phase(s) and/or for one or more cathodic stimulus phase(s). Transient blanking may be imposed for example for a period in the range of 10-50 μs either side of a stimulus transient. Noting that a stimulus phase width may be around 0.1-1 ms, such embodiments may thus provide for the measurement circuitry to be unblanked for 80-95% of the duration of each stimulus phase, while being blanked to avoid exposure to stimulus transients, allowing for evoked neural responses to be observed for a significant portion of the stimulus period while avoiding non-linearity, clipping or saturation of the measurement circuitry.

To permit observation of a neural response to commence prior to cessation of the stimulus, the measurement circuitry 128 is preferably unblanked or activated immediately following a stimulus feature which is expected to cause neural activation, such as the leading edge of a cathodic portion of the stimulus. For example, the measurement circuitry may be unblanked or activated within 50 μs, more preferably 20 μs, more preferably 10 μs, after such a stimulus feature.

Some embodiments may provide for a stimulus protocol to be applied whereby stimuli are delivered at high frequency and low current, in which a single stimulation is not expected to elicit a neural response but the temporal summation of several stimulations is intended to recruit an ECAP. Such embodiments may provide for the stimulus protocol to be halted once an ECAP is observed or once the ECAP amplitude, peak width or other characteristic reaches a threshold.

In some embodiments, the detection/measurement of the ECAP may be carried out in parallel on more than one recording electrode, to improve signal detection due to the spatial and temporal variations which will occur.

Some embodiments may provide for a stimulus intensity, such as stimulus current and/or stimulus voltage, to be progressively raised from a sub-threshold level so as to search for an ECAP recruitment threshold.

The neuromodulation may comprise spinal cord stimulation, sacral nerve stimulation, deep brain stimulation (DBS), vagus nerve stimulation, or other form of neuromodulation.

The method may be applied in respect of a single stimulus applied in isolation, or in respect of multiple stimuli applied repeatedly, sporadically, or continuously, such as at less than 10 Hz, at tens of Hz or at hundreds of Hz.

Some embodiments may comprise DBS monitoring of beta band oscillations, whereby the intra-stimulus response is measured continuously. For example DBS may be applied at tens or hundreds of Hz, and beta band oscillations variations may be computed and compared to changes of stimulus intensity or frequency or the like.

The stimulus may comprise a continuous or piecewise continuous waveform, wherein responses evoked by the continuous waveform can be used to adjust ongoing application of the waveform.

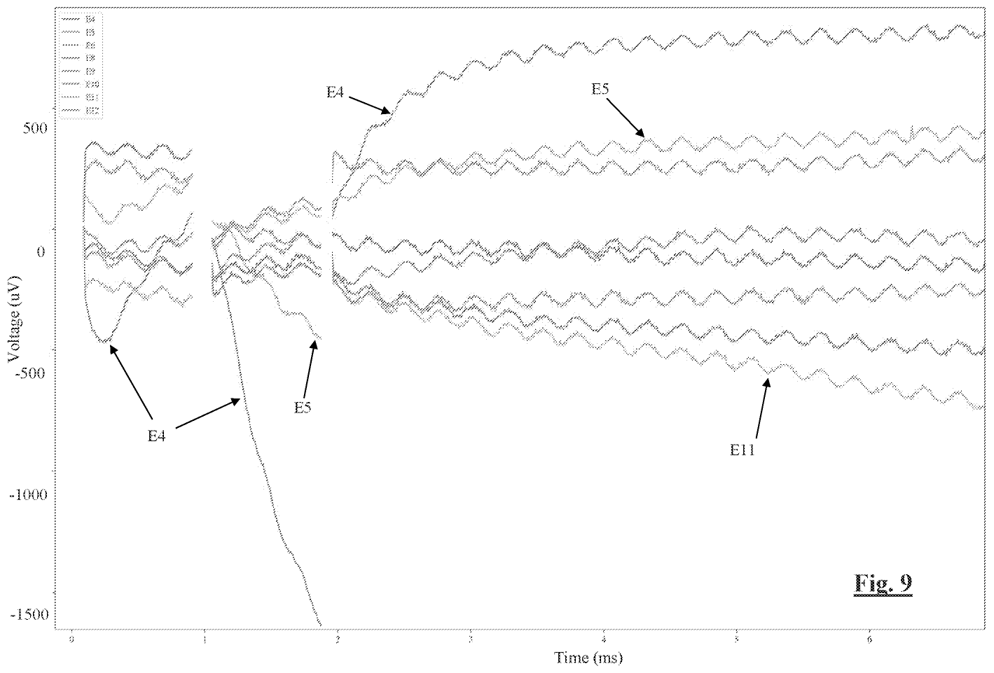

FIG. 8 illustrates the component waveforms as may arise in some embodiments of the invention. The signal on a recording electrode consists of a stimulus waveform with passive recovery (a) plus the ECAP (b) to give the composite waveform (c). Measuring the neural response may be done on the same electrode as is delivering the stimulation, or may be done on a nearby non-stimulating electrode. When the system detects the ECAP amplitude reaches the threshold (the feedback target), stimulation is ceased. The passive recovery waveform allows the system to automatically adjust for the varying pulse width of stimulation. An active charge recovery phase driven by a current source and providing matched charge could also be used.

By leaving a gap between the stimulating and charge-recovery pulses, the non-overlapping part of the ECAP can be recorded without interference.

To illustrate the ability of recording ECAPs during application of a stimulus, recording was performed experimentally, in sequence, on each of a number of electrodes of an electrode array. The results are shown in FIG. 9. The biphasic stimulus pulse lasts for approximately 1.8 ms with a phase transition at approximately 0.8 ms. The absence of data at certain times in each trace is because recording is suspended for approximately 70 μs at every current transition in the stimulus. No recordings were obtained for E1 or E2 (the stimulating electrodes), nor E3, nor E7 (the reference electrode). This is in accordance with transient blanking as described above.

Periods where the measurement circuitry prevented the amplifier from recording input are blanked in FIG. 9. The voltage arising on electrode E4 remains between about +700 μV and −1600 μV, and is thus kept within the maximum input range of 2.4 mV by the measurement circuitry. All other recordings remain within an even smaller range. In addition to the sinusoidal signal of interest (injected to simulate ECAPs), some undesirable residual stimulus artefact remains on the electrodes, particularly E4 and E5, as seen in the decaying excursions of these recordings. However, as these unwanted artefact components are kept within the input range of the amplifier chain, such unwanted components can be removed digitally via DSP techniques if required. As desired, the 50 μV sinusoidal 4 kHz signal being used to simulate ECAPs can be observed and thus easily retrieved from all recordings. Indeed, for the recordings from E6 and E8-E12 the sinusoidal signal of interest can be directly resolved without further processing, during the first 2 ms of the plot, i.e. during application of the stimulus. Characteristics of an ECAP arising in this manner in such recordings can thus be extracted and utilised to control or alter application of the same stimulus.

Some embodiments of the present invention thus recognise that an ability to record the neural response during application of the stimulus can provide for single-stimulus ECAP-controlled neuromodulation. This may thus provide a method and device for delivering a stimulus to neural tissue, where the amplitude (or other characteristic(s)) of the stimulus is controlled by observing the neural response the stimulus pulse itself is generating or has generated.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not limiting or restrictive.

Label list

| | |
|---|---|
| spinal cord stimulator | 100 |
| electronics module | 110 |
| battery | 112 |
| telemetry module | 114 |
| controller | 116 |
| memory | 118 |
| patient settings | 120 |
| control programs | 122 |
| pulse generator | 124 |
| electrode selection module | 126 |
| measurement circuitry | 128 |
| amplifier | 128a |
| digital converter | 128b |
| analog controller | 130 |
| recording electrode array | 150 |
| nerve | 180 |
| transcutaneous communication | 190 |
| external device | 192 |
| period | 402 |
| stimulus | 502 |
| cathodic portion | 504 |
| time | 506 |
| first ECAP recording | 508 |
| ECAP amplitude | 510 |
| stimulus | 522 |
| amplitude | 525 |
| second recording | 528 |
| third stimulus | 542 |
| time | 610 |
| time | 620 |
| neural response | 625 |
| amplitude | 630 |
| stimulus cessation time | 640 |
| method | 700 |
| step | 710 |
| step | 720 |
| step | 730 |
| step | 740 |
| step | 750 |
| step | 760 |
| step | 770 |
| step | 780 |
| step | 790 |
| step | 795 |

1. A device for recording evoked neural responses, the device comprising:
a plurality of electrodes including one or more stimulus electrodes and one or more sense electrodes;
a stimulus source for providing a stimulus to be delivered from the one or more stimulus electrodes to a neural pathway in order to give rise to an evoked compound action potential (ECAP) on the neural pathway;
measurement circuitry for recording a neural compound action potential signal sensed at the one or more sense electrodes, the measurement circuitry being operable to record the neural compound action potential signal during delivery of at least part of the stimulus; and
a control unit configured to process the recording of the neural compound action potential signal to determine at least one characteristic of the ECAP, the control unit further configured to define at least one characteristic of the stimulus based on the determined at least one characteristic of the ECAP.

2. The device of claim 1 wherein the at least one characteristic of the ECAP reflects an efficacy of the stimulus.

3. The device of claim 1 wherein the at least one characteristic of the ECAP comprises a binary characteristic.

4. The device of claim 3, wherein the at least one characteristic of the ECAP comprises one of:
an indication as to whether or not the stimulus has recruited an ECAP; and
a comparison of the ECAP to a threshold.

5. The device of claim 1 wherein the at least one characteristic of the ECAP comprises a gradated or scalar indication of an observed feature of the signal.

6. The device of claim 5, wherein the at least one characteristic of the ECAP comprises one or more of:
a measure of ECAP onset delay time;
a measure of ECAP slope;
a measure of ECAP amplitude such as ECAP peak amplitude;
a measure of ECAP duration such as ECAP peak width, ECAP zero crossing spacing, or ECAP half height width; and
a measure of ECAP spectral components.

7. The device of claim 1, wherein the control unit is further configured to cease recording of the neural compound action potential signal at a time defined relative to detection of a threshold, or at a time defined relative to when the stimulus ceases.

8. The device of claim 1, wherein the control unit is further configured to continue recording of the neural compound action potential signal after the step of defining at least one characteristic of the stimulus is completed.

9. The device of claim 1, wherein the control unit is further configured to determine the at least one characteristic of the ECAP prior to cessation of the stimulus, and to further use the determined at least one characteristic of the ECAP to control the manner in which a remainder of the stimulus is applied.

10. The device of claim 9, wherein the control unit is further configured to use the determined at least one characteristic of the ECAP to alter at least one stimulus parameter in order to control the stimulus to deliver a desired dose of neural recruitment.

11. The device of claim 10, wherein the control unit is configured to control the stimulus to deliver a desired dose of neural recruitment by controlling an amount of charge delivered to the neural pathway by the stimulus.

12. The device of claim 10, wherein the control unit is configured to control the stimulus to deliver a desired dose of neural recruitment by controlling one of a duration, an amplitude, an intensity, a voltage, a current and a morphology of the stimulus.

13. The device of claim 1, wherein the control unit is configured to define at least one characteristic of the stimulus by defining a number of pulses of the stimulus.

14. The device of claim 1, wherein the control unit is further configured to recover any imbalance of charge that occurred as a result of defining at least one characteristic of the stimulus.

15. The device of claim 1, wherein the control unit is configured to define at least one characteristic of the stimulus based on a determined at least one characteristic of at least one previous ECAP.

16. The device of claim 1, wherein one or more of the one or more stimulus electrodes also serve as one or more of the one or more sense electrodes.

17. The device of claim 1, wherein the one or more sense electrodes are non-stimulating electrodes.

18. The device of claim 1, wherein the control unit is further configured to blank the measurement circuitry during one or more stimulus transients.

19. The device of claim 18, wherein the control unit is further configured to unblank the measurement circuitry immediately following a stimulus feature which is expected to cause neural activation.

20. A method for recording evoked neural responses, the method comprising:
delivering a stimulus from one or more stimulus electrodes to a neural pathway in order to give rise to an evoked compound action potential (ECAP) on the neural pathway;
recording with measurement circuitry, during delivery of at least part of the stimulus, a neural compound action potential signal sensed at one or more sense electrodes;
processing the recording of the neural compound action potential signal to determine at least one characteristic of the ECAP; and
defining at least one characteristic of the stimulus based on the determined at least one characteristic of the ECAP.

21. The method of claim 20, wherein the at least one characteristic of the ECAP reflects an efficacy of the stimulus.

22. The method of claim 20 wherein the at least one characteristic of the ECAP comprises a binary characteristic.

23. (canceled)

24. The method of claim 20 wherein the at least one characteristic of the ECAP comprises a gradated or scalar indication of an observed feature of the signal.

25. (canceled)

26. The method of claim 20, further comprising ceasing recording of the neural compound action potential signal at a time defined relative to detection of a threshold, or at a time defined relative to when the stimulus ceases.

27. The method of claim 20, further comprising continuing recording of the neural compound action potential signal after the step of defining at least one characteristic of the stimulus is completed.

28. The method of claim 20, further comprising determining the at least one characteristic of the ECAP prior to cessation of the stimulus, and using the determined at least one characteristic of the ECAP to control the manner in which a remainder of the stimulus is applied.

29. (canceled)

30. (canceled)

31. (canceled)

32. (canceled)

33. (canceled)

34. The method of claim 20, wherein defining at least one characteristic of the stimulus is based on a determined at least one characteristic of at least one previous ECAP.

35. (canceled)

36. (canceled)

37. The method of claim 20, further comprising blanking the measurement circuitry during one or more stimulus transients.

38. (canceled)

39. A non-transitory computer readable medium for recording evoked neural responses, comprising instructions which, when executed by one or more processors, causes:
delivering a stimulus from one or more stimulus electrodes to a neural pathway in order to give rise to an evoked compound action potential (ECAP) on the neural pathway;
recording with measurement circuitry, during delivery of at least part of the stimulus, a neural compound action potential signal sensed at one or more sense electrodes;
processing the recording of the neural compound action potential signal to determine at least one characteristic of the ECAP; and
defining at least one characteristic of the stimulus based on the determined at least one characteristic of the ECAP.
